(19) United States
(12) Patent Application Publication
(10) Pub. No.: US 2023/0321062 A1
(43) Pub. Date: Oct. 12, 2023

(54) COMBINATION THERAPIES

(71) Applicant: Mirati Therapeutics, Inc., San Diego, CA (US)

(72) Inventor: Hirak Der-Torossian, San Diego, CA (US)

(21) Appl. No.: 18/026,799

(22) PCT Filed: Sep. 15, 2021
(86) PCT No.: PCT/US21/50447

§ 371 Date: Mar. 16, 2023

Related U.S. Application Data
(60) Provisional application No. 63/179,896, filed on Apr. 26, 2021.
(60) Provisional application No. 63/079,883, filed on Sep. 17, 2020.

(51) Int. Cl. A61K 31/444; A61K 39/395; A61K 47/68; A61P 35/00

(52) U.S. Cl. CPC A61K 31/444; A61K 39/3955; A61K 47/6803; A61P 35/00; A61K 2039/507

(57) ABSTRACT

The present invention relates to combination therapies for treating cancer, in particular a multityrosine kinase-associated cancer. The present invention provides therapeutically effective combinations of: a) N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); plus b) a PD-(L)1/PD-1 checkpoint inhibitor; and/or c) the Nectin-4 directed antibody-drug conjugate; as well as methods of using these combinations to treat cancer.

### FIELD OF THE INVENTION

The present invention relates to combination therapies useful for treating cancer. In particular, the present invention relates to therapeutically effective combinations of: a) N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); b) a PD-(L)1/PD-1 checkpoint inhibitor; and c) a Nectin-4 directed antibody drug conjugate (ADC).

### BACKGROUND OF THE INVENTION

International publication No. WO2009/026717A disclosed compounds with the inhibition activities of multiple protein tyrosine kinases, for example, the inhibition activities of VEGF receptor kinase and HGF receptor kinase. In particular, disclosed N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide (sitravatinib) is a multi-tyrosine kinase inhibitor with demonstrated potent inhibition of a closely related spectrum of tyrosine kinases, including RET, CBL, CHR4q12, DDR and Trk, which are key regulators of signaling pathways that lead to cell growth, survival and tumor progression.

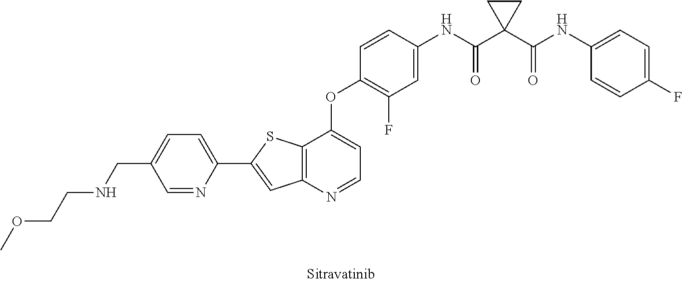 [SMILES: COCCNCc1ccc(-c2cc3nccc(Oc4ccc(NC(=O)C5(C(=O)Nc6ccc(F)cc6)CC5)cc4F)c3s2)nc1]

Sitravatinib shows tumor regression in multiple human xenograft tumor models in mice, and is presently in human clinical trials.

Immune checkpoint inhibitor therapy using antibodies blocking T cell negative regulatory molecules, such as CTLA-4 and PD-1, has delivered positive results in some cancer patients. However, due to the complex network of immunosuppressive pathways present in advanced tumors, only a minority of patients respond to this therapy. Efforts to sensitize tumors to immune checkpoint inhibitor therapy are ongoing.

One example of a checkpoint inhibitor is pembrolizumab. Pembrolizumab is a monoclonal antibody that binds to the human PD-1 receptor and blocks its interaction with PD-L1 and PD-L2, thereby releasing PD-1 pathway-mediated inhibition of the immune response, including the anti-tumor immune response. Pembrolizumab, sequences thereof, and methods of making and using this antibody, including for increasing the activity of an immune response through the PD-1 pathway, are disclosed WO 2008/156712.

Pembrolizumab is the active pharmaceutical ingredient in Keytruda®, which has been approved by the FDA for the treatment of melanoma, NSCLC in a PD-L1 positive patient population, and squamous cell carcinoma of the head and neck. More specifically with regard to NSCLC, Keytruda® (pembrolizumab) is approved in the US for patients with metastatic NSCLC whose tumors express PD-L1 as determined by an FDA-approved test and who have disease progression on or after platinum-containing chemotherapy.

A Nectin-4 directed antibody-drug conjugate (ADC) comprises an antibody directed against Nectin-4 conjugated to an auristatin (for example, monomethyl auristatin E (MMAE); monomethyl auristatin F (MMAF); PF-06380101 or others). Auristatins are a family of complex analogues to the native antineoplastic product dolastatin 10. They are 100 to 1000 times more toxic than Doxorubicin, a conventional cancer chemotherapy medication. In the ADC, the auristatin is attached to the antibody via a protease cleavable linker.

One example of such an ADC is enfortumab vedotin. Enfortumab vedotin (sometimes referred to as simply “enfortumab”) is an ADC comprised of a fully human anti Nectin-4 IgG1 monoclonal antibody conjugated to MMAE via a protease-cleavable linker. Nectin-4, also known as poliovirus receptor-related protein 4 (PVRL4), is an adhesion protein located on the surface of cells, with weak to moderate expression in normal skin. Copy number gain of the PVRL4 gene is a frequent event in carcinogenesis and promotes epithelial to mesenchymal transition, invasion and metastasis, resulting in high expression of Nectin-4 in several solid tumors, particularly urothelial carcinomas. Enfortumab binds to cells that express Nectin-4 with high affinity, triggering the internalization and release of MMAE in target cells, inducing cell cycle arrest and apoptotic cell death. Enfortumab has been approved under accelerated approval based on tumor response rate for the treatment of adult patients with locally advanced or metastatic urothelial cancer who have previously received a PD-(L)1/PD-1 checkpoint inhibitor, and a platinum-based chemotherapy in the neoadjuvant/adjuvant, locally advanced or metastatic setting.

Enfortumab vedotin is the active pharmaceutical ingredient in PADCEV®, which has been approved by the FDA and is indicated for the treatment of adult patients with locally advanced or metastatic urothelial cancer (mUC) who have previously received a programmed death receptor-1 (PD-1) or programmed death-ligand 1 (PD-L1) inhibitor, and a platinum-containing chemotherapy in the neoadjuvant/adjuvant, locally advanced or metastatic setting.

There is a need in the art to arrive at new and improved methods of treating cancer.

### SUMMARY OF THE INVENTION

The combination therapy of the present invention, in one aspect, synergistically increases the potency of sitravatinib resulting in improved therapeutic efficacy. The combination therapy of the present invention, in another aspect, provides improved clinical benefit to patients compared to treatment with the disclosed ingredients as monotherapies.

In one aspect, the invention provides a method of treating cancer in a subject in need thereof, comprising administering to the subject a therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); a PD-(L)1/PD-1/PD-1 checkpoint inhibitor; and a Nectin-4 directed antibody-drug conjugate (ADC).

In another aspect, the invention provides a method of treating cancer in a subject in need thereof, comprising administering to the subject a therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib) and a PD-(L)1/PD-1/PD-1 checkpoint inhibitor.

In still another aspect, the invention provides a method of treating cancer in a subject in need thereof, comprising administering to the subject a therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib) and a Nectin-4 directed antibody-drug conjugate (ADC).

In one aspect of the invention, the PD-(L)1/PD-1 checkpoint inhibitor is pembrolizumab.

In one aspect of the invention, the Nectin-4 directed ADC is enfortumab vedotin.

In one aspect of the invention, the cancer is a multi-tyrosine kinase-associated cancer. In one embodiment, the multi-tyrosine kinase-associated cancer is selected from the group consisting of bladder cancer, including urothelial carcinoma; lung cancer, including non-small cell lung cancer (NSCLC); kidney cancer, including renal cell carcinoma; head and neck cancer, including squamous cell carcinoma; ovarian cancer; stomach cancer; and liver cancer, including hepatocellular carcinoma.

In one aspect of the invention, sitravatinib is administered orally once per day.

In one aspect of the invention, the therapeutically effective amount of sitravatinib is between about 35 mg and about 100 mg.

In one aspect of the invention, pembrolizumab is administered by intravenous (IV) infusion once every three weeks.

In one aspect of the invention, the therapeutically effective amount of pembrolizumab is about 200 mg.

In one aspect of the invention, enfortumab vedotin is administered by IV infusion twice every three weeks.

In one aspect of the invention, the therapeutically effective amount of enfortumab vedotin is between about 1 mg/kg and about 1.25 mg/kg.

### DETAILED DESCRIPTION OF THE INVENTION

The present invention relates to combination therapies for treating cancer, in particular a multi-tyrosine kinase-associated cancer. In one aspect, the present invention relates to therapeutically effective combinations of: a) N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); b) a PD-(L)1/PD-1 checkpoint inhibitor; and c) a Nectin-4 directed ADC.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer, in particular a multi-tyrosine kinase-associated cancer (e.g., as determined using a regulatory agency-approved, e.g., FDA-approved, assay or kit). In some embodiments, the subject is suspected of having particular a multi-tyrosine kinase-associated cancer.

The term “regulatory agency” is a country's agency for the approval of the medical use of pharmaceutical agents with the country. For example, a non-limiting example of a regulatory agency is the U.S. Food and Drug Administration (FDA).

As used herein, “an effective amount” of a compound is an amount that is sufficient to negatively modulate or inhibit the activity of the desired target. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of receptor tyrosine kinases (RTKs). Such amount may be administered as a single dosage or may be administered according to a regimen, whereby it is effective.

As used herein, a “therapeutically effective amount of a combination” of multiple compounds is an amount that together synergistically increases the activity of the combination in comparison to the therapeutically effective amount of each compound in the combination, i.e., more than merely additive.

In vivo, the therapeutically effective amount of the combination of a) N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); b) a PD-(L)1/PD-1 checkpoint inhibitor; and c) the Nectin-4 directed antibody drug conjugate, or pharmaceutically acceptable salts or pharmaceutical compositions thereof, results in an increased duration of overall survival (“OS”) in subjects relative to treatment with only sitravatinib or only the PD-(L)1/PD-1 checkpoint inhibitor or only enfortumab vedotin. In one embodiment, the therapeutically effective amount of the combination of a) sitravatinib; b) a PD-(L)1/PD-1 checkpoint inhibitor; and c) the Nectin-4 directed antibody drug conjugate, or pharmaceutically acceptable salts or pharmaceutical compositions thereof, results in an increased duration of progression-free survival (“PFS”) in subjects relative to treatment with only sitravatinib or only the PD-(L)1/PD-1 checkpoint inhibitor or only the Nectin-4 directed antibody drug conjugate. In one embodiment, the therapeutically effective amount of the combination of a) sitravatinib; b) a PD-(L)1/PD-1 checkpoint inhibitor; and c) the Nectin-4 directed antibody drug conjugate, or pharmaceutically acceptable salts or pharmaceutical compositions thereof, results in increased tumor regression in subjects relative to treatment with only sitravatinib or only the PD-(L)1/PD-1 checkpoint inhibitor or only the Nectin-4 directed antibody drug conjugate. In one embodiment, the therapeutically effective amount of the combination of a) sitravatinib; b) a PD-(L)1/PD-1 checkpoint inhibitor; and c) the Nectin-4 directed antibody drug conjugate, or pharmaceutically acceptable salts or pharmaceutical compositions thereof, results in increased tumor growth inhibition in subjects relative to treatment with only sitravatinib or only the PD-(L)1/PD-1 checkpoint inhibitor or only the Nectin-4 directed antibody drug conjugate. In one embodiment, the therapeutically effective amount of the combination of a) sitravatinib; b) a PD-(L)1/PD-1 checkpoint inhibitor; and c) the Nectin-4 directed antibody drug conjugate, or pharmaceutically acceptable salts or pharmaceutical compositions thereof, results in an improvement in the duration of stable disease in subjects compared to treatment with only sitravatinib or only the PD-(L)1/PD-1 checkpoint inhibitor or only the Nectin-4 directed antibody drug conjugate.

Alternatively, in vivo, the therapeutically effective amount of the combination of a) N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); and c) a Nectin-4 directed antibody drug conjugate, or pharmaceutically acceptable salts or pharmaceutical compositions thereof, results in an increased duration of overall survival (“OS”) in subjects relative to treatment with only sitravatinib or only enfortumab vedotin. In one embodiment, the therapeutically effective amount of the combination of a) sitravatinib; and c) the Nectin-4 directed antibody drug conjugate, or pharmaceutically acceptable salts or pharmaceutical compositions thereof, results in an increased duration of progression-free survival (“PFS”) in subjects relative to treatment with only sitravatinib or only the Nectin-4 directed antibody drug conjugate. In one embodiment, the therapeutically effective amount of the combination of a) sitravatinib; and c) the Nectin-4 directed antibody drug conjugate, or pharmaceutically acceptable salts or pharmaceutical compositions thereof, results in increased tumor regression in subjects relative to treatment with only sitravatinib or only the Nectin-4 directed antibody drug conjugate. In one embodiment, the therapeutically effective amount of the combination of a) sitravatinib; and c) the Nectin-4 directed antibody drug conjugate, or pharmaceutically acceptable salts or pharmaceutical compositions thereof, results in increased tumor growth inhibition in subjects relative to treatment with only sitravatinib or only the Nectin-4 directed antibody drug conjugate. In one embodiment, the therapeutically effective amount of the combination of a) sitravatinib; and c) the Nectin-4 directed antibody drug conjugate, or pharmaceutically acceptable salts or pharmaceutical compositions thereof, results in an improvement in the duration of stable disease in subjects compared to treatment with only sitravatinib or only the Nectin-4 directed antibody drug conjugate.

Alternatively, in vivo, the therapeutically effective amount of the combination of a) N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide (aka sitravatinib); and b) a PD-(L)1/PD-1 checkpoint inhibitor; or pharmaceutically acceptable salts or pharmaceutical compositions thereof, results in an increased duration of overall survival (“OS”) in subjects relative to treatment with only sitravatinib or only the PD-(L)1/PD-1 checkpoint inhibitor. In one embodiment, the therapeutically effective amount of the combination of a) sitravatinib; and b) a PD-(L)1/PD-1 checkpoint inhibitor; or pharmaceutically acceptable salts or pharmaceutical compositions thereof, results in an increased duration of progression-free survival (“PFS”) in subjects relative to treatment with only sitravatinib or only the PD-(L)1/PD-1 checkpoint inhibitor. In one embodiment, the therapeutically effective amount of the combination of a) sitravatinib; b) a PD-(L)1/PD-1 checkpoint inhibitor; or pharmaceutically acceptable salts or pharmaceutical compositions thereof, results in increased tumor regression in subjects relative to treatment with only sitravatinib or only the PD-(L)1/PD-1 checkpoint inhibitor. In one embodiment, the therapeutically effective amount of the combination of a) sitravatinib; b) a PD-(L)1/PD-1 checkpoint inhibitor; or pharmaceutically acceptable salts or pharmaceutical compositions thereof, results in increased tumor growth inhibition in subjects relative to treatment with only sitravatinib or only the PD-(L)1/PD-1 checkpoint inhibitor. In one embodiment, the therapeutically effective amount of the combination of a) sitravatinib; b) a PD-(L)1/PD-1 checkpoint inhibitor; or pharmaceutically acceptable salts or pharmaceutical compositions thereof, results in an improvement in the duration of stable disease in subjects compared to treatment with only sitravatinib or only the PD-(L)1/PD-1 checkpoint inhibitor.

The amount of each compound in the combination may be the same or different than the therapeutically effective amount of each compound when administered alone as a monotherapy as long as the combination is synergistic. Such amounts may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of any of the active ingredients or a pharmaceutically acceptable salt thereof, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

### Inhibitor Compounds

In one aspect of the invention, provided herein are methods of treating cancer in a subject in need thereof, comprising administering to the subject a therapeutically effective amount of a combination of sitravatinib, a PD-(L)1/PD-1 checkpoint inhibitor, e.g. pembrolizumab, and the Nectin-4 directed antibody drug conjugate, e.g. enfortumab vedotin.

### 1. Sitravatinib (N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide)

Sitravatinib (MGCD516) is an orally available, potent small molecule inhibitor of a closely related spectrum of receptor tyrosine kinases (RTKs) including MET, Axl, MERTK, VEGFR family, PDGFR family, KIT, FLT3, Trk family, RET, DDR2, and selected Eph family members.

The chemical structure, formula, and molecular weight of sitravatinib (MGCD516) free base and malate salt are as follows:

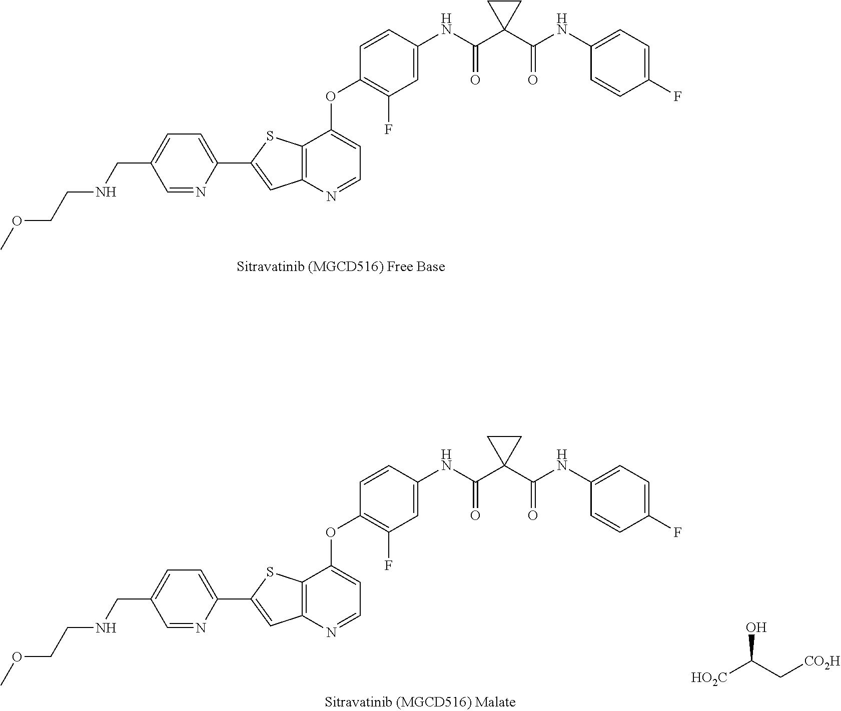 [SMILES: COCCNCc1ccc(-c2cc3nccc(Oc4ccc(NC(=O)C5(C(=O)Nc6ccc(F)cc6)CC5)cc4F)c3s2)nc1 ; COCCNCc1ccc(-c2cc3nccc(Oc4ccc(NC(=O)C5(C(=O)Nc6ccc(F)cc6)CC5)cc4F)c3s2)nc1 ; O=C(O)C[C@H](O)C(=O)O]

Chemical Formula: MGCD516 Free Base: C₃₃H₂₉F₂N₅O₄S
   MGCD516 Malate: C₃₇H₃₅F₂N₅O₉S
Molecular Weight: MGCD516 Free Base: 629.68
   MGCD516 Malate: 763.76

Receptor tyrosine kinases (RTKs) are key regulators of signaling pathways leading to cell growth, survival, and migration. These kinases are dysregulated in many cancers through overexpression, genetic alteration or co-expression with high affinity ligands. Multiple sitravatinib RTK targets are genetically altered in a variety of cancers and act as oncogenic drivers, promoting cancer development and progression. In addition to the immunostimulatory effects of Axl and MET inhibition, sitravatinib may further condition the MET in favor of antitumor activity by its immunomodulatory effects mediated through VEGFR and KIT inhibition. Preclinical data with sitravatinib indicate that it can increase expression of PD-L1 on tumor cells in vitro and in vivo. Pilot studies in syngeneic mouse tumor models also suggest that sitravatinib increases the proliferation and fraction of systemic/spleen CD4+ and CD8+T lymphocytes and reduces the number of systemic MDSCs. Additional studies to investigate the effects of sitravatinib in the tumor microenvironment are ongoing or planned.

### Nonclinical Data for Sitravatinib

Sitravatinib demonstrated potent, concentration-dependent inhibition of the kinase activity of MET, Axl, MERTK, VEGFR family, PDGFR family, KIT, FLT3, Trk family, RET, DDR2, and selected Eph family members in biochemical assays and inhibited phosphorylation and kinase dependent function in cell-based assays. Sitravatinib also inhibited oncogenic functions associated with target RTKs including MET-dependent cell viability and migration and endothelial tube formation and angiogenesis. Consistent with this anti-tumor and anti-angiogenic mechanism of action, sitravatinib demonstrated anti tumor efficacy over a broad spectrum of human tumor xenograft models including robust cytoreductive anti-tumor activity in a subset of models exhibiting genetic alterations in RTK targets including MET, RET, FLT3 and others.

In vitro results from the (human Ether-a-go-go Related Gene) hERG assay demonstrate an IC50 of 0.6 μM on the potassium current, which far exceeds exposures observed clinically. There were no adverse effects on the cardiovascular system, including no effect on the QTc interval, when sitravatinib was administered to dogs at doses up to 4 mg/kg (mean 6 hr concentration of 0.072 μg/mL). Minor increases in vascular pressures were observed during the dog cardiovascular study; however, these were mild and considered of limited biological consequence. Assessment of the neurological functional observation battery and respiratory evaluations (tidal volume, respiration rate, and minute volume) in rats did not reveal any sitravatinib related effects at doses up to 25 mg/kg.

In a bidirectional permeability study with Caco-2 cell lines, sitravatinib is classified as a highly permeable compound, and not a substrate of P-glycoprotein (P-gp) and Breast Cancer Resistance Protein (BCRP). A P-gp and BCRP inhibition study using Caco-2 cells suggested that MGCD516 is a significant inhibitor of P-gp and BCRP with IC50 value of 0.838 and 1.51 μM, respectively, these values are much higher than the systemic steady state exposure levels observed clinically.

Using an ultra-centrifugation technique sitravatinib was 98.6% bound to human plasma proteins.

Sitravatinib (MGCD516) was evaluated for cytochrome P-450-mediated metabolism using human liver microsomes and recombinant human enzymes. Results suggest that multiple enzymes, including CYP 1A2, 2A6, 2B6, 2C8, 2C9, 2D6, 2E1, and 3A4 are involved in the metabolism of sitravatinib.

The effect of treating primary cultures of cryopreserved human hepatocytes with MGCD516 on the expression of cytochrome P450 (CYP) enzymes was investigated. Overall, treatment of cultured human hepatocytes with up to 30 μM MGCD516 caused little or no increase (<2.0-fold change or <20% of the positive control) in CYP1A2 activity, CYP1A2 mRNA levels, or CYP3A4 activity. However, MGCD516 (up to 3 and 10 μM) caused concentration dependent increases (>2-fold change and >20% of the positive control) in CYP2B6 activity, CYP2B6 mRNA levels, and CYP3A4 mRNA levels in one or more human hepatocyte cultures.

There was little or no evidence of direct inhibition of CYP1A2, CYP2A6 or CYP2E1 by MGCD516 or time- or metabolism-dependent inhibition of any of the CYP enzymes evaluated. Under the experimental conditions examined, MGCD516 demonstrated direct inhibition of CYP2C8, CYP2C9, CYP2C19, CYP2D6 and CYP3A4/5 (as measured by testosterone 60-hydoxylation and midazolam 1′-hydroxylation) with IC50 values of 2.9 μM, 11 μM, 10 μM, 1.9 μM, 11 μM and 0.81 μM, respectively. In addition, approximate 50% direct inhibition was observed for CYP2B6 at the highest concentration of MGCD516 evaluated (20 μM); thus, the IC50 value was reported as greater than 20 μm.

Because the potency for MGCD516 against its intended clinical targets is generally less than 0.1 μM, it may be unlikely that concentrations required for robust direct systemic inhibition/induction of the tested CYPs will be achieved at projected clinical dose and exposure levels.

### Sitravatinib Pharmacokinetics (PK)

After single dose administration of sitravatinib free base capsules, sitravatinib reaches peak concentration in a median time of 3 to 8 hours. Exposure parameters (maximum concentration [Cmax] and area under the curve [AUC]) are dose proportional with doses up to 200 mg. Mean elimination half life varies between 42 and 58 hours after oral administration.

We previously evaluated the relative bioavailability and PK of sitravatinib in plasma following single doses of sitravatinib free base and sitravatinib malate capsule formulations in healthy subjects in a 2-part, open-label, crossover study (Study 516-006).

Study 516-006 was a Phase 1, 2-part, open-label, single-dose, crossover study designed to evaluate the relative bioavailability of sitravatinib free base and sitravatinib malate capsule formulations in healthy subjects. In each part, subjects were randomized into 2 treatment sequences (either test then reference or reference then test formulations) and participated in two 7-day treatment periods separated by a washout period. Part 1 assessed the relative bioavailability and PK of a single oral dose of 80 mg sitravatinib administered as free base capsule formulation (reference product) and malate capsule formulation (test product). From Part 1, it was determined that the malate capsule formulation is statistically significantly more bioavailable than the free base capsule formulation, and that a free base to malate ratio of approximately 1.25 would give similar PK exposure. Subsequently, Part 2 assessed the relative bioavailability and PK of a single oral dose of 120 mg sitravatinib free base capsule formulation (reference product) and 100 mg sitravatinib malate capsule formulation (test product). The administration of 100 mg malate capsule formulation compared to 120 mg free base capsule formulation were similar based on descriptive statistics; for 100 mg malate capsule formulation vs. 120 mg free base capsule formulation, the geometric mean AUC0 ∞, AUC0-t and Cmax was 3074 vs. 3089 ng*h/mL, 2943 vs. 2962 ng*h/mL and 56.4 vs. 55.1 ng/mL, respectively. The inferential statistical analysis showed that the ratio and 90% confidence interval of the geometric least squares means of AUC0 ∞, AUC0 t and Cmax were within the regulatory acceptance range of 80-125%, demonstrating that the 120 mg sitravatinib free base and 100 mg malate capsule formulations are bioequivalent.

In the instant study, in Part 1, we compared the same single dose of 80 mg sitravatinib as free base and malate capsule formulations. Geometric mean Cmax, AUC0-∞ and AUC0-168 was 1.27-, 1.24- and 1.24 fold higher following malate capsules administration compared to free base capsules administration. From Part 1, it was determined that the malate capsule formulation was statistically significantly more bioavailable than the free base formulation, and that a free base to malate ratio of approximately 1.25 would give similar PK exposure.

In Part 2, malate capsule formulation dose was adjusted and the geometric mean Cmax was comparable (55.1 and 56.4 ng/mL, respectively) following single dose administration of 120 mg sitravatinib free base formulation and a lower 100 mg sitravatinib dose of sitravatinib malate capsule formulation. The geometric mean AUC0-168 was 2962 and 2943 ng*h/mL for 120 mg sitravatinib free base and 100 mg sitravatinib malate capsule formulations, respectively. The geometric mean t½ was similar following malate capsule formulation administration compared to free base capsule formulation administration, with estimates being 35.0 and 34.3 hours, respectively, and individual t½ values ranging from 25.4 to 52.0 hours and from 23.2 to 55.4 hours, respectively. Inferential statistical analysis showed that the ratio and 90% confidence interval of the geometric least squares (LS) means of AUC0-∞, AUC0-t and Cmax were 98.9 [91.8, 106.6], 98.8 [91.6, 106.5] and 102.4% [92.9, 112.7], respectively. Study 516-006 demonstrated bioequivalence between the 120 mg sitravatinib free base and 100 mg sitravatinib malate capsule formulations.

### 2. PD-(L)1/PD-1 Checkpoint Inhibitors, e.g. Pembrolizumab

Immune checkpoint inhibitor therapy using antibodies blocking T cell negative regulatory molecules, such as CTLA-4 and PD-1, has delivered positive results in some cancer patients. However, due to the complex network of immunosuppressive pathways present in advanced tumors, only a minority of patients respond to this therapy. Efforts to sensitize tumors to immune checkpoint inhibitor therapy are ongoing.

One example of a checkpoint inhibitor is pembrolizumab. Pembrolizumab is a monoclonal antibody that binds to the human PD-1 receptor and blocks its interaction with PD-L1 and PD-L2, thereby releasing PD-1 pathway-mediated inhibition of the immune response, including the anti-tumor immune response. Pembrolizumab, sequences thereof, and methods of making and using this antibody, including for increasing the activity of an immune response through the PD-1 pathway, are disclosed WO 2008/156712.

Pembrolizumab (KEYTRUDA©) is a humanized monoclonal antibody that blocks the interaction between PD-1 and its ligands, PD-L1 and PD-L2. Pembrolizumab is an IgG4 kappa immunoglobulin.

Binding of the PD-1 ligands, PD-L1 and PD-L2, to the PD-1 receptor found on T cells, inhibits T-cell proliferation and cytokine production. Upregulation of PD-1 ligands occurs in some tumors and signaling through this pathway can contribute to inhibition of active T-cell immune surveillance of tumors. Pembrolizumab is a monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

### Pembrolizumab Pharmacokinetics (PK)

The PK of pembrolizumab was characterized using a population PK analysis with concentration data collected from 2993 patients with various cancers who received pembrolizumab doses of 1 to 10 mg/kg every two weeks (Q2W), 2 to 10 mg/kg every three weeks (Q3W), or 200 mg Q3W. The geometric mean (% coefficient of variation [CV %]) clearance (CL) is 195 mL/d (40%), geometric mean volume of distribution at steady state (Vss) is 6.0 L (20%), and geometric mean terminal elimination half-life (ti/2) is 22 days (32%). Steady state concentrations of pembrolizumab were reached by 16 weeks of repeated dosing with a Q3W regimen, and systemic accumulation was approximately 2.1-fold. The steady-state exposure to pembrolizumab increased dose proportionally over the dose range of 2 to 10 mg/kg administered Q3W.

The population PK analysis suggested that the following factors had no clinically important effect on the clearance of pembrolizumab: age (15 to 94 years), sex, race (89% White), renal impairment (eGFR ≥15 mL/min/1.73 m2), mild hepatic impairment (total bilirubin [TB] less than or equal to the upper limit of normal [ULN] and AST greater than ULN or TB<1 to 1.5 times ULN or AST>ULN), or tumor burden. The impact of moderate or severe hepatic impairment on the PK of pembrolizumab is unknown.

### Pembrolizumab Efficacy Reported in Urothelial Carcinoma Clinical Trials

In the KEYNOTE-045 study (Bellmunt-2017; Fradet-2019), the median duration of therapy in the pembrolizumab group was 3.5 months (range, <0.1 to 20.0) and the median follow-up was 27.7 months. The overall ORR as confirmed by central review in the pembrolizumab group was 21% (20% for patients with CPS≥10). The median DOR was not reached (range, 1.6+ to 30.0+ months). Median overall survival was 10.1 months. Pembrolizumab continued to demonstrate an OS benefit over chemotherapy in all subgroups examined, including those with visceral disease and liver metastases, and across the different levels of PD-L1 expression and risk groups.

In the KEYNOTE-052 study (Balar-2017; O'Donnell-2017; O'Donnell-2019), the overall ORR as confirmed by central review was 29%, compared to 47% for patients with CPS ≥10. With a median follow-up of 15.3 months, the median DOR was 30.1 months (range, 18.8 months to not reached) in the overall population; and was not reached for patients with CPS ≥10. Median overall survival was 11.3 months (range, 9.7 to 13.1 months), compared to 18.5 months (range, 12.2 to 28.5 months) for patients with CPS ≥10.

### 3. Nectin-4 Directed Antibody Drug Conjugate (ADC), e.g., Enfortumab Vedotin

Nectin-4 is an adhesion protein located on the surface of cells. A Nectin-4 directed antibody-drug conjugate (ADC) comprises an antibody directed against Nectin-4 conjugated to an auristatin (for example, monomethyl auristatin E (MMAE); monomethyl auristatin F (MMAF); PF-06380101 or others). Auristatins are a family of complex analogues to the native antineoplastic product dolastatin 10. They are 100 to 1000 times more toxic than Doxorubicin, a conventional cancer chemotherapy medication. In the ADC, the auristatin is attached to the antibody via a protease cleavable linker.

In one aspect of the invention, the ADC is enfortumab vedotin. Enfortumab vedotin-ejfv (enfortumab, PADCEV™) is a Nectin-4 directed ADC comprised of a fully human anti-Nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline linker (SGD-1006).

Nonclinical data suggest that the anticancer activity of enfortumab vedotin-ejfv is due to the binding of the ADC to Nectin-4-expressing cells, followed by internalization of the ADC-Nectin-4 complex, and the release of MMAE via proteolytic cleavage. Release of MMAE disrupts the microtubule network within the cell, subsequently inducing cell cycle arrest and apoptotic cell death.

### Enfortumab Pharmacokinetics

Population pharmacokinetic analysis included data from 369 patients based on three Phase 1 studies and one Phase 2 study. Enfortumab pharmacokinetics were characterized after single and multiple doses in patients with locally advanced or metastatic urothelial carcinoma (UC) and other solid tumors. Enfortumab exhibited linear dose-proportional PK at doses ranging from 0.5 to 1.25 mg/kg when administered as an intravenous infusion over −30 minutes on days 1, 8, and 15 of a 28-day cycle in patients with locally advanced or metastatic UC. Peak enfortumab concentrations were attained at the end of infusion. In contrast, plasma concentrations of free MMAE increased until ˜2 days after enfortumab dosing. The mean clearance (CL) of enfortumab and free MMAE in patients was 0.10 L/h and 2.7 L/h, respectively, in patients. Elimination of MMAE appeared to be limited by its rate of release from enfortumab. The estimated mean volume of distribution of ADC at steady state (Vss) was 11 liters following administration of enfortumab. ADC and MMAE exhibited multi-exponential declines with an elimination half-life of 3.4 days and 2.4 days, respectively. Steady-state concentrations of ADC and MMAE were reached after 1 treatment cycle with a 1.25 mg/kg Q3W regimen, and minimal accumulation of the ADC and MMAE was observed following repeat administration of enfortumab in patients.

Based on population pharmacokinetic analysis, no clinically significant differences in the pharmacokinetics of enfortumab were observed based on age (24 to 87 years), sex, or race/ethnicity (Caucasian, Asian, Black, or others).

Based on population pharmacokinetics analysis, there was a 48% AUC increase in unconjugated MMAE exposure observed in patients with mild hepatic impairment (bilirubin of 1 to 1.5 the upper limit of normal [ULN] and AST less than ULN, or bilirubin less than or equal to ULN and AST greater than ULN, n=31) compared to normal hepatic function. The effect of moderate or severe hepatic impairment (AST or ALT greater than 2.5 times ULN or total bilirubin greater than 1.5 times ULN) or liver transplantation on the pharmacokinetics of ADC or unconjugated MMAE is unknown.

The pharmacokinetics of enfortumab and MMAE were evaluated after the administration of 1.25 mg/kg of enfortumab to patients with mild (creatinine clearance; CrCL greater than 60 to 90 mL/min; n=135), moderate (CrCL 30 to 60 mL/min; n=147) and severe (CrCL less than 30 mL/min; n=8) renal impairment. No significant differences in exposure (AUC) of ADC and MMAE were observed in patients with mild, moderate or severe renal impairment compared to patients with normal renal function. The effect of end stage renal disease with or without dialysis on the pharmacokinetics of ADC or unconjugated MMAE is unknown.

### Enfortumab Efficacy Reported in Urothelial Carcinoma Clinical Trials

In the EV-201 study (Petrylak-2019; Rosenberg-2019), the median duration of therapy was 4.6 months (maximum duration of 15.6 months) and the median follow-up was 10.2 months. The overall ORR as confirmed by central review was 44% (including a 12% complete response rate). The median DOR was 7.6 months (range, 0.95 to 11.30+ months). Median overall survival was 11.7 months.

In the EV-103 study (Rosenberg-2020; Hoimes-2019), the median follow-up was 10.4 months. The overall ORR as confirmed by central review was 73% (including a 16% complete response rate) with activity regardless of PD-L1 expression level. With a median follow-up was 10.4 months, the median DOR was not reached (range, 1.2 to 12.9+ months). Median overall survival was also not reached, with a 12-month overall survival rate of 82%.

### Pharmaceutical Compositions

In another aspect, the invention provides pharmaceutical compositions comprising sitravatinib; b) a PD-(L)1/PD-1 checkpoint inhibitor; and c) a Nectin-4 directed antibody drug conjugate (ADC) according to the invention and a pharmaceutically acceptable carrier, excipient, or diluent that may be used in the methods disclosed herein. Sitravatinib, a PD-(L)1/PD-1 checkpoint inhibitor; and a Nectin-4 directed ADC may be formulated by any method well known in the art and may be prepared for administration by any route, including, without limitation, parenteral, oral, sublingual, transdermal, topical, intranasal, intratracheal, intravenous or intrarectal. In certain embodiments, a PD-(L)1/PD-1 checkpoint inhibitor and a Nectin-4 directed ADC are administered intravenously in a hospital setting. In one embodiment, administration of sitravatinib may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising the ingredients of the provided combination may be used in the methods of use described herein.

### Co-Administration

Sitravatinib, a PD-(L)1/PD-1 checkpoint inhibitor; and a Nectin-4 directed ADC can be formulated into separate or individual dosage forms which can be co-administered one after the other. If the route of administration is the same (e.g. oral), the active compounds can be formulated into a single form for co-administration.

The pharmaceutical compositions comprising sitravatinib, a PD-(L)1/PD-1 checkpoint inhibitor; and a Nectin-4 directed ADC for use in the methods may be for simultaneous, separate or sequential use. In one embodiment, sitravatinib is administered orally once per day continuously in 21-day cycles (for example, at 35 mg, 50 mg, 70 mg, or 100 mg).

In one embodiment, a PD-(L)1/PD-1 checkpoint inhibitor (for example, pembrolizumab) is administered through intravenous (IV) infusion once every 3 weeks (for example, at 200 mg over 30 minutes).

In one embodiment, a Nectin-4 directed ADC (for example, enfortumab vedotin) is administered through IV infusion twice every 3 weeks (for example, on Day 1 and Day 8 in 21-day cycles; for example, at 1 mg/kg or 1.25 mg/kg).

In some embodiments, separate administration of each inhibitor, at different times and by different routes, in some cases may be advantageous.

Oncology drugs are typically administered at the maximum tolerated dose (“MTD”), which is the highest dose of drug that does not cause unacceptable side effects. In one embodiment, sitravatinib, a PD-(L)1/PD-1 checkpoint inhibitor; and a Nectin-4 directed ADC are each dosed at their respective MTDs. In one embodiment, sitravatinib is dosed at its MTD and a PD-(L)1/PD-1 checkpoint inhibitor; and a Nectin-4 directed ADC are each dosed in an amount less than their respective MTDs. In one embodiment, sitravatinib is dosed at an amount less than its MTD and a PD-(L)1/PD-1 checkpoint inhibitor and/or a Nectin-4 directed ADC are each dosed at their respective MTDs. In one embodiment, sitravatinib, a PD-(L)1/PD-1 checkpoint inhibitor; and a Nectin-4 directed ADC are each dosed at less than their respective MTDs. The administration can be so timed that the peak pharmacokinetic effect of one compound coincides with the peak pharmacokinetic effect of one or both of the other compounds.

In one embodiment, sitravatinib and a PD-(L)1/PD-1 checkpoint inhibitor are each dosed at their respective MTDs. In one embodiment, sitravatinib is dosed at its MTD and a PD-(L)1/PD-1 checkpoint inhibitor is dosed in an amount less than its MTDs. In one embodiment, sitravatinib is dosed at an amount less than its MTD and a PD-(L)1/PD-1 checkpoint inhibitor is dosed at its MTD. In one embodiment, sitravatinib and a PD-(L)1/PD-1 checkpoint inhibitor are each dosed at less than their respective MTDs. The administration can be so timed that the peak pharmacokinetic effect of one compound coincides with the peak pharmacokinetic effect of one or both of the other compounds.

In one embodiment, sitravatinib and a Nectin-4 directed ADC are each dosed at their respective MTDs. In one embodiment, sitravatinib is dosed at its MTD and a Nectin-4 directed ADC is dosed in an amount less than its MTD. In one embodiment, sitravatinib is dosed at an amount less than its MTD and a Nectin-4 directed ADC are each dosed at their respective MTDs. In one embodiment, sitravatinib and a Nectin-4 directed ADC are each dosed at less than their respective MTDs. The administration can be so timed that the peak pharmacokinetic effect of one compound coincides with the peak pharmacokinetic effect of one or both of the other compounds.

In one embodiment, a single dose of sitravatinib is administered per day (i.e., in about 24 hour intervals) (i.e., QD).

In one embodiment, a single dose of a PD-(L)1/PD-1 checkpoint inhibitor (for example, pembrolizumab) is administered through IV infusion once every 3 weeks.

In one embodiment, a single dose of a Nectin-4 directed ADC is administered through IV infusion twice every 3 weeks.

### Combination Therapies

In one aspect of the invention, provided herein are methods of treating cancer in a subject in need thereof, comprising administering to the subject a therapeutically effective amount of sitravatinib, a PD-(L)1/PD-1 checkpoint inhibitor, and a Nectin-4 directed antibody-drug conjugate (ADC).

In one embodiment, the invention provides methods of treating cancer in a subject in need thereof, comprising administering to the subject a therapeutically effective amount of sitravatinib and a PD-(L)1/PD-1 checkpoint inhibitor. In such an embodiment, the PD-(L)1/PD-1 checkpoint inhibitor may be pembroluzimab.

In one embodiment, the invention provides methods of treating cancer in a subject in need thereof, comprising administering to the subject a therapeutically effective amount of sitravatinib and a Nectin-4 directed antibody-drug conjugate (ADC). In such an embodiment, the Nectin-4 directed antibody-drug conjugate (ADC) may be enfortumab vedotin.

In one aspect of the invention, the cancer is a multi-tyrosine kinase-associated cancer. In one embodiment, the multi-tyrosine kinase-associated cancer is selected from the group consisting of bladder cancer, including urothelial carcinoma; lung cancer, including non-small cell lung cancer (NSCLC); kidney cancer, including renal cell carcinoma; head and neck cancer, including squamous cell carcinoma; ovarian cancer; stomach cancer; and liver cancer, including hepatocellular carcinoma.

In one embodiment, the therapeutically effective amount of the combination of sitravatinib, a PD-(L)1/PD-1 checkpoint inhibitor, and a Nectin-4 directed antibody-drug conjugate (ADC), or pharmaceutically acceptable salts or pharmaceutical compositions thereof, results in an increased duration of overall survival (“OS”) in subjects relative to treatment with only sitravatinib or only one of the other two active ingredients.

In one embodiment, the therapeutically effective amount of the combination of sitravatinib, a PD-(L)1/PD-1 checkpoint inhibitor, and a Nectin-4 directed ADC, or pharmaceutically acceptable salts or pharmaceutical compositions thereof, results in an increased duration of progression-free survival (“PFS”) in subjects relative to treatment with only sitravatinib or only one of the other two active ingredients.

In one embodiment, the therapeutically effective amount of the combination of sitravatinib, a PD-(L)1/PD-1 checkpoint inhibitor, and a Nectin-4 directed ADC, or pharmaceutically acceptable salts or pharmaceutical compositions thereof, results in increased tumor regression in subjects relative to treatment with only sitravatinib or only one of the other two active ingredients.

In one embodiment, the therapeutically effective amount of the combination of sitravatinib, a PD-(L)1/PD-1 checkpoint inhibitor, and a Nectin-4 directed ADC, or pharmaceutically acceptable salts or pharmaceutical compositions thereof, results in increased tumor growth inhibition in subjects relative to treatment with only sitravatinib or only one of the other two active ingredients.

In one embodiment, the therapeutically effective amount of the combination of sitravatinib, a PD-(L)1/PD-1 checkpoint inhibitor, and a Nectin-4 directed ADC, or pharmaceutically acceptable salts or pharmaceutical compositions thereof, results in an improvement in the duration of stable disease in subjects compared to treatment with only sitravatinib or only one of the other two active ingredients.

In another embodiment, the PD-(L)1/PD-1 checkpoint inhibitor and a Nectin-4 directed ADC are administered in combination with sitravatinib once disease progression has been observed for sitravatinib monotherapy, in which the combination therapy results in enhanced clinical benefit for the patient by increasing OS, PFS, tumor regression, tumor growth inhibition or the duration of stable disease in the patient.

In one embodiment, sitravatinib is administered as a capsule during the period of time. In one embodiment, a tablet or capsule formulation of sitravatinib comprises between about 35 mg to about 100 mg.

In one embodiment, the amount of sitravatinib is selected from the group consisting of about 35 mg, about 50 mg, about 70 mg and about 100 mg.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

### EXAMPLES OF THE INVENTION

### Example 1: A Combinational Treatment of Patients with Sitravatinib, Pembrolizumab and Enfortumab

### Formulation of Sitravatinib

The composition of the drug product used in previous clinical trials consists of a blend of sitravatinib (MGCD516) free base drug substance, microcrystalline cellulose, polysorbate 80, and colloidal silicon dioxide. The blend is filled into hard gelatin capsules. To help differentiate between the products, the free base formulation will be labeled with the drug product code “MGCD516”.

The sitravatinib malate capsule product consists of a blend of sitravatinib malate drug substance, microcrystalline cellulose, mannitol, croscarmellose sodium, colloidal silicon dioxide, and magnesium stearate; the blend is filled into hard gelatin capsules. To help differentiate between the products, the malate formulation will be labeled with the drug product name “Sitravatinib”.

The strengths of all sitravatinib capsule formulations are expressed based on sitravatinib free base weight. Dose strengths of sitravatinib and additional packaging differences between formulations will be provided in the Pharmacy Study Manual.

Sitravatinib drug product is packaged in high-density polyethylene (HDPE), white opaque, round 60 cc bottles. A tamper proof heat induction seal and a child resistant closure are used. The provided bottles may be labeled for specific patient use and given to the patient.

### Formulation of Pembrolizumab

Pembrolizumab is obtained from commercial sources and managed in accordance with known procedures.

### Formulation of Enfortumab

Enfortumab is obtained from commercial sources and managed in accordance with known procedures.

### Example 2: Patient Groups for the Combinational Therapy

Patient Group 1

Patient Group 1 are patients that were previously treated with checkpoint inhibitor and platinum-based chemotherapy. There are 2 parts to this Patient Group: a lead-in dose escalation portion and a dose expansion portion. In the dose escalation portion, patients are treated with up to 3 dose levels of sitravatinib in combination with up to 2 dose levels of pembrolizumab and enfortumab combination regimen to determine the recommended doses to be used in the combination treatment regimen and those doses will be further studied in the dose expansion portion.

The dosing is as follows: pembrolizumab 200 mg over 30 min IV infusion every 3 weeks, sitravatinib orally once per day continuously in 21-day cycles (at 35 mg, 50 mg, 70 mg, or 100 mg) and enfortumab vedotin 8 in 21-day cycles (at 1 mg/kg or 1.25 mg/kg).

Patient Group 2

Patient Group 2 starts enrollment after Patent Group 1 (dose-escalation portion) has determined the recommended doses for the regimen. Patient Group 2 consists of patients with previously untreated unresectable, locally advanced or metastatic unrothelial cancer.

The dosing is as follows: pembrolizumab 200 mg over 30 min IV infusion every 3 weeks, sitravatinib orally once per day continuously in 21-day cycles (recommended dose from Patient Group 1) and enfortumab vedotin over 30 min IV infusion on Day 1 and Day 8 in 21-day cycles (recommended dose from Patient Group 1).

### Example 3: Administration of the Drugs

Sitravatinib capsules may be administered orally, once daily (QD), in a continuous regimen expressed in 21-day cycles for the sitravatinib, pembrolizumab and enfortumab combination Patient Groups.

For the sitravatinib, pembrolizumab and enfortumab combination Patient Groups, the sitravatinib starting dose for the lead-in dose escalation portion of Patient Group 1 using the malate capsule formulation may be 35 mg QD which represents three dose levels below the dose administered as a single agent and in combination (with nivolumab) Phase 2 and Phase 3 trials. If a dose is identified for sitravatinib in combination with pembrolizumab and enfortumab, this recommended starting dose may be used for subsequent contingent sitravatinib, pembrolizumab and enfortumab combinations (dose expansion portion of Patient Group 1 and Patient Group 2). Capsules may be taken on an empty stomach (at least 2-hour fast before each dose and no food for a minimum of 1 hour after each dose). The fasting requirement may be eliminated based on the outcome of an ongoing food-effect study under a separate study protocol.

On days when pembrolizumab or enfortumab are administered, the daily dose of sitravatinib may precede the pembrolizumab or enfortumab infusion for logistical reasons.

Available dose levels for each of the sitravatinib formulations are outlined in Table 1 and Table 2. Dose modifications guidelines for the malate capsule formulation (Table 2) follow comparable dose reduction proportions used for the free base capsule formulation. Once the dose has been reduced, re-escalation is generally not recommended but may be considered on a case-by-case basis with approval from the Medical Monitor. If the administration of sitravatinib is interrupted for reasons other than toxicity, then treatment with the study drug may be resumed at the same dose.

The starting dose for sitravatinib using the free base capsule formulation is 120 mg QD.

TABLE 1
Sitravatinib Sequential Dose Reductions for Individual
Patients on Free Base Capsule Formulation
120 mg once daily
80 mg once daily
60 mg once daily
40 mg once daily

The starting dose for sitravatinib using the malate capsule formulation is 100 mg QD.

TABLE 2
Sitravatinib Sequential Dose Reductions for Individual
Patients on Malate Capsule Formulation
100 mg once daily
70 mg once daily
50 mg once daily
35 mg once daily
20 mg once daily*
*This dose level is only available for Patient Groups 1 and 2.

In the event of sitravatinib-related AE, dose reduction with continuous treatment is preferred over repeated dose interruption. If treatment with sitravatinib is delayed for ≥14 days, then resumption at a reduced dose may be considered. If treatment with sitravatinib is withheld for ≥28 consecutive days, then permanent discontinuation of sitravatinib may be considered.

Pembrolizumab (KEYTRUDA) is administered as an intravenous infusion over approximately 30 minutes (+/−5 minutes) at 200 mg every 3 weeks (Q3W). Pembrolizumab should be administered after sitravatinib (and specifically after the 30 minute PK sampling on applicable study visits), and at least 30 minutes before enfortumab.

Enfortumab (PADCEV) may be administered in this study as an intravenous infusion over approximately 30 minutes (+/−5 minutes) at the selected dose level on Days 1, and 8 of a 21-day cycle, at the discretion of the Investigator and in accordance with the current PADCEV US Prescribing Information. Note: enfortumab should be administered at least 30 minutes after pembrolizumab.

Dosing is based on patient weight. Enfortumab may be administered at mg/kg doses based on the patient's actual body weight at Cycle 1 Day 1. For on-study dosing, the dose may be adjusted if the patient's weight changes by ≥10% from their Cycle 1 Day 1 weight, or if enfortumab dose modification criteria are met. Other dose adjustments for changes in body weight <10% are permitted per institutional standard. For patients who weigh >100 kg, the dose may be calculated based on 100 kg weight. Institutional dose rounding rules may be applied to this protocol, but otherwise, doses of the investigational product may be rounded to the nearest milligram.

Patients receive enfortumab administration only if their laboratory results of blood drawn within 24 hours prior to dosing meets the following study drug administration laboratory criteria:

Blood glucose ≤250 mg/dL.

### Example 4: Evaluating Sitravatinib Combinations with a PD-(L)1/PD-1 Checkpoint Inhibitor and an Antibody-Drug Conjugate

Study Design—Design details specific to Patient Group 1 (including lead-in dose escalation and dose expansion portions) and Patient Group 2 are described in Example 2 and below, including description of the populations to be enrolled into these contingent Patient Groups evaluating the triple combination of sitravatinib, pembrolizumab and enfortumab, the study design and dose-limiting toxicities (DLTs) as applicable.

Initially, the lead-in dose escalation portion of Patient Group 1 may evaluate sitravatinib administered in combination with pembrolizumab and enfortumab in patients who have previously received a PD-(L)1/PD-1 checkpoint inhibitor and a platinum-based chemotherapy. If a tolerable dose is identified for sitravatinib in combination with pembrolizumab and enfortumab, the recommended dose regimen of sitravatinib in combination with pembrolizumab and enfortumab may be further evaluated in as many as 2 populations including in patients who have previously received a PD-(L)1/PD-1 checkpoint inhibitor and a platinum-based chemotherapy (dose expansion portion of Patient Group 1), and in patients with previously untreated locally advanced or metastatic urothelial cancer (Patient Group 2). Description of the populations to be enrolled into Patient Group 1 and Patient Group 2 evaluating the triple combination of sitravatinib, pembrolizumab and enfortumab are described below.

Patient Group 1 (including lead-in dose escalation and dose expansion portions): Patients who have previously received a PD-(L)1/PD-1 checkpoint inhibitor and a platinum-containing chemotherapy.

Patient Group 2: Patients with previously untreated unresectable, locally advanced or metastatic urothelial cancer.

Lead-In Dose Escalation Portion of Patient Group 1 Evaluating Sitravatinib in Combination with Pembrolizumab and Enfortumab

This portion of the study begins with the lead-in dose escalation portion of Patient Group 1 of up to three dose levels of sitravatinib in combination with up to two dose levels of pembrolizumab and enfortumab combination regimen, in patients who have previously received a PD-(L)1/PD-1 checkpoint inhibitor and a platinum-based chemotherapy. Sitravatinib starting dose levels using the malate formulation are shown in Table 3, whereas the starting doses for pembrolizumab and enfortumab are shown in Table 4. Dosing begins at 35 mg QD of sitravatinib in combination with the recommended doses of pembrolizumab and enfortumab combination regimen. This starting dose for sitravatinib represents three dose levels below the dose administered as a single agent and in combination (with nivolumab) Phase 2 and 3 trials. Dose Level 3 in Table 3 is the dose used in single agent and combination Phase 2 and 3 trials. The starting doses for pembrolizumab and enfortumab (labeled as Dose Level 1 in Table 4) represent the recommended doses from the EV-103 Phase 2 trial and the EV-302 Phase 3 trial of pembrolizumab and enfortumab used in combination (Rosenberg-2020; Hoimes-2019). It is of note that the EV-302 Phase 3 trial also uses these recommended doses of pembrolizumab and enfortumab in the triple combination investigation arm combining pembrolizumab and enfortumab with the platinum-chemotherapy agents cisplatin or carboplatin. In addition, a dose de-escalation step for enfortumab may be undertaken as appropriate (labeled as Dose Level −1 in Table 4). Throughout the study, pembrolizumab and enfortumab are administered in accordance with approved labeling. Guidance for adverse event management and associated pembrolizumab treatment modifications are provided in product labeling.

TABLE 3
Sitravatinib Starting Dose Levels for Lead-In Dose
Escalation Patient Group 1 (Malate Formulation)

| Sitravatinib Dose Level | Sitravatinib Daily Dose |
|---|---|
| 1 | 35 mg PO QD |
| −2¹ | 50 mg PO QD |
| 2 | 70 mg PO QD |
| 3 | 100 mg PO QD |

Abbreviations: PO = orally; QD = once daily.

The 50 mg dose level may be enrolled only if de-escalation is needed after assessment of the next higher dose level (70 mg).

TABLE 4
Pembrolizumab and Enfortumab Dose Levels

| Combination | | |
|---|---|---|
| Regimen | | |
| Dose Level | Pembrolizumab¹ | Enfortumab¹ |
| −1² | 200 mg IV Q3W | 1 mg/kg IV on days 1 and 8 of Q3W |
| 1 | 200 mg IV Q3W | 1.25 mg/kg IV on days 1 and 8 of Q3W |

Abbreviations: IV = intravenous; Q3W = every 3 weeks.
¹Pembrolizumab and enfortumab should be administered according to USPI and standard care.
²The 1 mg/kg dose level of enfortumab will be enrolled only if de-escalation is needed after assessment of the next higher dose level (1.25 mg/kg).

Dose-Limiting Toxicities (DLTs) in Cycle 1 for the Lead-In Dose Escalation Portion of Patient Group 1

DLTs are only defined for patients in the lead-in dose escalation portion of Patient Group 1. The definition of DLT for the purpose of dose escalation decisions includes any of the following events considered to be causally related to treatment with sitravatinib in combination with pembrolizumab and enfortumab that occurs from Cycle 1 Day 1 through pre-dose Cycle 2 Day 1:

Hematological DLTs:
Grade 4 neutropenia, if it lasts more than 7 days.
≥Grade 3 febrile neutropenia.
≥Grade 3 neutropenia with significant clinical sequelae.
Grade 4 thrombocytopenia.
Grade 3 thrombocytopenia associated with clinically significant bleeding.
Any requirement for a platelet transfusion.
Grade 4 anemia unexplained by underlying disease.

Non-hematological DLTs:
≥Grade 4 infusion related reaction.
Grade 3 infusion related reaction that does not resolve within 24 hours.
Any ≥Grade 4 non-hematological toxicity.
Grade 3 hypertension that cannot be controlled with medical therapy, including:
   Severe hypertension with systolic blood pressure ≥180 mmHg or diastolic blood pressure ≥120 mmHg, on more than one occasion.
   Sustained uncontrolled hypertension, with systolic blood pressure ≥160 mmHg (but <180 mmHg) or diastolic blood pressure ≥100 mmHg (but <120 mmHg) lasting for ≥14 days or causing treatment delay for ≥4 days.
Other Grade 3 non-hematologic toxicity lasting for >3 days despite optimal supportive care, with the following exceptions:
   Endocrine disorder (thyroid, pituitary, and/or adrenal insufficiency) that can be effectively managed with hormone replacement therapy.
   Fatigue that persists for ≤7 days.
   Rash that resolves to Grade ≤1 within 3 weeks.
   Tumor flare (defined as local pain, irritation, or rash localized at sites of known or suspected tumor).
Grade 2 pneumonitis or colitis that does not resolve to ≤Grade 1 as indicated by symptoms within 3 days after onset of the event despite optimal medical management with or without corticosteroids.
≥Grade 3 non-hematological laboratory abnormalities with clinical consequences that do not resolve within 24 hours.
ALT >3×ULN with bilirubin >2×ULN.
Any other related toxic effect during the first 3 weeks may be assessed as a DLT if, upon review by the Investigators and Sponsor, it is agreed that the toxicity was of sufficient severity to be considered dose limiting.
Any toxicity that requires suspension of sitravatinib for more than 1 week.
A patient did not receive at least 1 dose of pembrolizumab and at least 2 doses of enfortumab up to and including Cycle 2 Day 1 due to tolerability issues (i.e., other related AEs not attributed as DLTs).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of treating cancer in a subject in need thereof, comprising administering to the subject a therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide; a PD-(L)1/PD-1 checkpoint inhibitor; and a Nectin-4 directed antibody-drug conjugate (ADC).

2. A method of treating cancer in a subject in need thereof, comprising administering to the subject a therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide; and a PD-(L)1/PD-1 checkpoint inhibitor.

3. A method of treating cancer in a subject in need thereof, comprising administering to the subject a therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide; and a Nectin-4 directed antibody-drug conjugate (ADC).

4. The method of claim 1, wherein the PD-(L)1/PD-1 checkpoint inhibitor is pembrolizumab.

5. The method of claim 1, wherein the Nectin-4 directed antibody-drug conjugate is enfortumab vedotin.

6. The method of claim 1, wherein the cancer is a multi-tyrosine kinase-associated cancer.

7. The method of claim 6, wherein the multi-tyrosine kinase-associated cancer is selected from the group consisting of bladder cancer, lung cancer, kidney cancer, head and neck cancer, ovarian cancer, stomach cancer, and liver cancer.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The method of claim 1, wherein N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide, the PD-(L)1/PD-1 checkpoint inhibitor, and the Nectin-4 directed antibody-drug conjugate are administered on the same day.

21. The method of claim 1, wherein N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide, the PD-(L)1/PD-1 checkpoint inhibitor, and the Nectin-4 directed antibody-drug conjugate are administered on different days.

22. The method of claim 1, wherein N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide is administered orally.

23. (canceled)

24. The method of claim 1, wherein the PD-(L)1/PD-1 checkpoint inhibitor is administered by intravenous infusion.

25. (canceled)

26. The method of claim 1, wherein the Nectin-4 directed antibody-drug conjugate is administered by intravenous infusion.

27. (canceled)

28. The method of claim 1 wherein N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide is administered at a maximum tolerated dose.

29. The method of claim 1, wherein the PD-(L)1/PD-1 checkpoint inhibitor is administered at a maximum tolerated dose.

30. The method of claim 1, wherein the Nectin-4 directed antibody-drug conjugate is administered at a maximum tolerated dose.

31. The method of claim 1, wherein N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide, the PD-(L)1/PD-1 checkpoint inhibitor, and the Nectin-4 directed antibody-drug conjugate are each administered at a maximum tolerated dose.

32. (canceled)

33. (canceled)

34. The method of claim 1, wherein the therapeutically effective amount of the combination of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide, the PD-(L)1/PD-1 checkpoint inhibitor, and the Nectin-4 directed antibody-drug conjugate results in an increased duration of overall survival, an increased duration of progression free survival, an increase in tumor growth regression, an increase in tumor growth inhibition or an increased duration of stable disease in the subjects relative to treatment with only N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide.

35. The method according to claim 1, wherein the therapeutically effective amount of N-(3-Fluoro-4-(2-(5-((2-methoxyethylamino)methyl)pyridin-2-yl)thieno[3,2-b]pyridin-7-yloxy) phenyl)-N-(4-fluorophenyl)cyclopropane-1,1-dicarboxamide is between about 35 mg and about 100 mg.

36. (canceled)

37. (canceled)

38. (canceled)

39. (canceled)

40. The method according to claim 1, wherein the therapeutically effective amount of the PD-(L)1/PD-1 checkpoint inhibitor is about 200 mg.

41. The method according to claim 1, wherein the therapeutically effective amount of the Nectin-4 directed antibody-drug conjugate is between about 1 mg/kg and about 1.25 mg/kg.

42. (canceled)

43. (canceled)
